(19) United States
(12) Patent Application Publication
(10) Pub. No.: US 2026/0090794 A1
(43) Pub. Date: Apr. 2, 2026

(54) CARDIAC BIOPSY DEVICES WITH SENSING AND ASSOCIATED METHODS

(71) Applicant: Medtronic, Inc., Minneapolis, MN (US)

(72) Inventor: Xiaohong Zhou, Woodbury, MN (US)

(21) Appl. No.: 19/337,983

(22) Filed: Sep. 24, 2025

Related U.S. Application Data
(60) Provisional application No. 63/701,434, filed on Sep. 30, 2024.

(51) Int. Cl. A61B 10/02; A61B 5/24

(52) U.S. Cl. CPC A61B 10/0233; A61B 5/24

(57) ABSTRACT

Devices and methods for collecting biopsy samples are provided. In some embodiments, a method includes introducing a biopsy device into a heart of a patient, where the biopsy device includes an elongate shaft having a distal end and a helical element at the distal end; generating sensor data indicative of a characteristic of cardiac tissue of the heart using at least one sensor coupled to or associated with the biopsy device; identifying a location of pathological tissue in the cardiac tissue based on the sensor data; and collecting a sample of the pathological tissue using the helical element.

### RELATED APPLICATIONS

This application claims the benefit of U.S. Provisional Application Ser. No. 63/701,434, filed Sep. 30, 2024, the entire contents of which are incorporated herein by reference.

### TECHNICAL FIELD

The present technology generally relates to medical devices, and in particular, to cardiac biopsy devices with sensing and associated methods.

### BACKGROUND

Endomyocardial biopsy (EMB) is a diagnostic tool for evaluating underlying causes of myocardial disease (e.g., cardiomyopathy, myocarditis, unexplained ventricular arrhythmias), cardiac involvement in systemic diseases, and cardiac allograft rejection. EMB is typically performed by sampling the right ventricular septum via the right internal jugular vein using guidance (e.g., fluoroscopy). Some clinical practices use a forceps-style cutting and grasping instrument known as a bioptome to obtain a sample of cardiac tissue during EMB. Use of a bioptome for EMB can increase the risk of myocardial perforation, ventricular arrhythmias, damage to the tricuspid valve, and venous access site related complications. Moreover, the relatively large size of the bioptome (e.g., 5-7 Fr) can make it difficult to navigate to the target site. Although a Medtronic SelectSure MRI SureScan Model 3830 pacing lead can potentially be used to perform EMB in the right ventricular septum, challenges remain in collecting clinically relevant tissue samples with minimal damage to healthy tissue.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates a procedure for collecting a cardiac tissue sample from a heart of a patient using a biopsy device, in accordance with embodiments of the present technology.

FIG. 2 is a partially schematic illustration of a biopsy device, in accordance with embodiments of the present technology.

FIGS. 3A-3D are partially schematic illustrations of various sensor configurations for the biopsy device of FIG. 2, in accordance with embodiments of the present technology.

FIG. 4 is a flow diagram illustrating a method for collecting a biopsy sample from a patient, in accordance with embodiments of the present technology.

FIGS. 5A-5C illustrate representative examples of waveforms corresponding to pathological cardiac activity, in accordance with embodiments of the present technology.

FIGS. 6A-6F illustrate representative examples of fragmented QRS waveforms corresponding to pathological cardiac activity, in accordance with embodiments of the present technology.

FIG. 7 is a flow diagram illustrating a method for collecting a biopsy sample from a patient, in accordance with embodiments of the present technology.

### DETAILED DESCRIPTION

The present technology relates to devices and methods for collecting tissue from a patient's body, such as a biopsy sample. In some embodiments, for example, a device for collecting a biopsy sample includes an elongate shaft having a proximal end and a distal end, where the distal end is configured to be introduced into a heart of a patient and positioned proximate to cardiac tissue of the heart, e.g., with or without assistance of a delivery catheter or other component that can navigate to the target site. The device can also include a helical element (e.g., a coil, screw, spring) at the distal end of the elongate shaft. The helical element can be configured to be advanced into the cardiac tissue to obtain a sample thereof. The device can further include at least one sensor (e.g., an electrical sensor and/or a mechanical sensor) configured to generate sensor data (e.g., electrical and/or mechanical signals) indicative of a characteristic of the cardiac tissue. The sensor data can be received and analyzed by a controller to determine whether the cardiac tissue includes pathological tissue. If pathological tissue is detected, the controller can instruct a user to collect a sample of the pathological tissue using the helical element. If no pathological tissue is detected, the controller can instruct a user to reposition the distal end of the elongate shaft to another location in the heart.

The present technology can provide numerous advantages compared to conventional devices and methods for EMB. For instance, bioptome devices used in conventional EMB procedures are relatively large, and thus may be challenging to navigate to the target site in the heart and may also cause tissue trauma during the navigation and biopsy process. Moreover, bioptome-based EMB performed during implantation of cardiac implantable electrical devices (CIEDs) has been shown to increase the risk of myocardial perforation, life-threatening ventricular arrhythmias, damage to the tricuspid valve, and venous access site-related complications. Thus, bioptome-based EMB is conventionally avoided during the periprocedural period of CIED implantation and post-lead implantation period in the right ventricle. Additionally, conventional EMB devices lack the capability to distinguish pathological tissue from healthy tissue, which makes it challenging for the surgeon to ensure that clinically relevant tissue is sampled and may lead to an inconclusive or inaccurate diagnosis of the patient's condition. Sampling of healthy tissue may be unhelpful for diagnostic purposes and may cause unnecessary trauma to the heart.

The present technology can overcome these and other challenges by using a helical element to collect tissue samples, which can reduce tissue trauma compared to conventional bioptomes that utilize a forceps-style collection element. The devices herein can also be smaller and more flexible than conventional bioptomes, thereby providing easier navigation and reducing the risk of inadvertent damage to cardiac structures. Moreover, the devices herein can incorporate sensing capabilities to allow clinically relevant tissue (e.g., pathological tissue) to be accurately identified, thereby enhancing the accuracy of the diagnostic procedure while also avoiding unnecessary damage to healthy tissue. The devices and methods herein can be used in a wide variety of applications where biopsy is desired, such as in patients who are candidates for receiving an implanted cardiac device and are indicated for EMB.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” “lower,” “left,” “right,” etc., can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

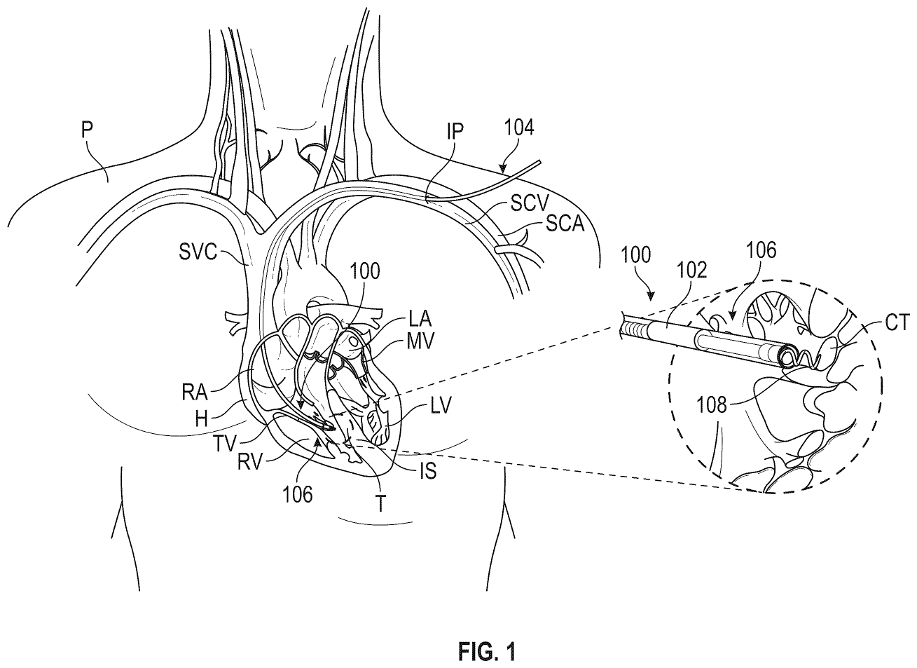

FIG. 1 illustrates a procedure for collecting a cardiac tissue sample from a heart H of a patient P using a biopsy device 100, in accordance with embodiments of the present technology. The biopsy device 100 can be configured to be navigated to a target region T (e.g., a myocardial wall, an interventricular septum IS, a septal wall, a heart valve) in the heart H. In some embodiments, to be proximate to a target region T, a portion of the biopsy device 100 (e.g., a distal end) is positioned within a heart chamber, such as within the right ventricle RV, within the right atrium RA, within the left atrium LA, or within the left ventricle LV. The biopsy device 100 can be positioned at any of a variety of locations in the heart H to sense and/or collect a sample of tissue therefrom. For example, as shown in FIG. 1, the biopsy device 100 can be inserted through an insertion point IP of a subclavian vein SCV, navigated within the vasculature to a superior vena cava SVC, enter the heart H through the right atrium RA, pass through a tricuspid valve TV into the right ventricle RV, and be positioned proximate to the target region T of the heart H of the patient P where cardiac tissue CT (e.g., ventricular myocardium, septal wall tissue) may be sensed and/or collected. Although FIG. 1 illustrates an example procedure in which the biopsy device 100 is navigated to the target region T in the right ventricle RV in the heart H via the insertion point IP of the subclavian vein SCV, it will be appreciated that other vascular access points and other vascular routes to a target region T of the heart H are possible (e.g., jugular vein, femoral vein, femoral artery).

Delivery of the biopsy device 100 to the target region T in the heart H may be achieved through many techniques. In some embodiments, the biopsy device 100 is navigated through a vascular route to the target region T in the heart H via a delivery catheter or sheath that surrounds at least a portion of the biopsy device 100 (e.g., a distal portion of the biopsy device 100). In some embodiments, the biopsy device 100 is navigated to the target region T in the heart H by advancing over a stylet or guidewire. In some embodiments, the biopsy device 100 is steered into proximity to the target region T in the heart H without requiring a separate device, e.g., via integral cables or other steering elements that are part of the biopsy device 100.

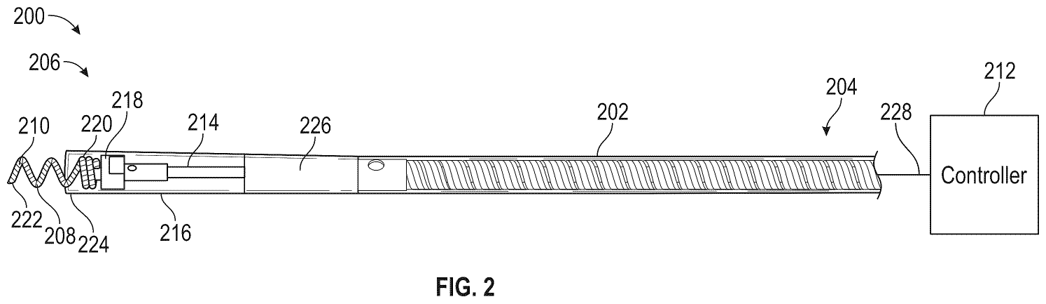

In the illustrated biopsy procedure of FIG. 1, the biopsy device 100 comprises an elongate shaft 102 with a proximal end 104 and a distal end 106, the distal end 106 configured to be positioned proximate to the target region T comprising cardiac tissue CT (e.g., ventricular myocardium, septal wall tissue). The distal end 106 can include a helical element 108 for collecting a sample of the cardiac tissue CT. For example, the helical element 108 can be advanced partially or entirely into the cardiac tissue CT, and then retracted relative to the cardiac tissue CT to remove a sample thereof. Additional details of the helical element 108 are provided below, e.g., in connection with FIG. 2.

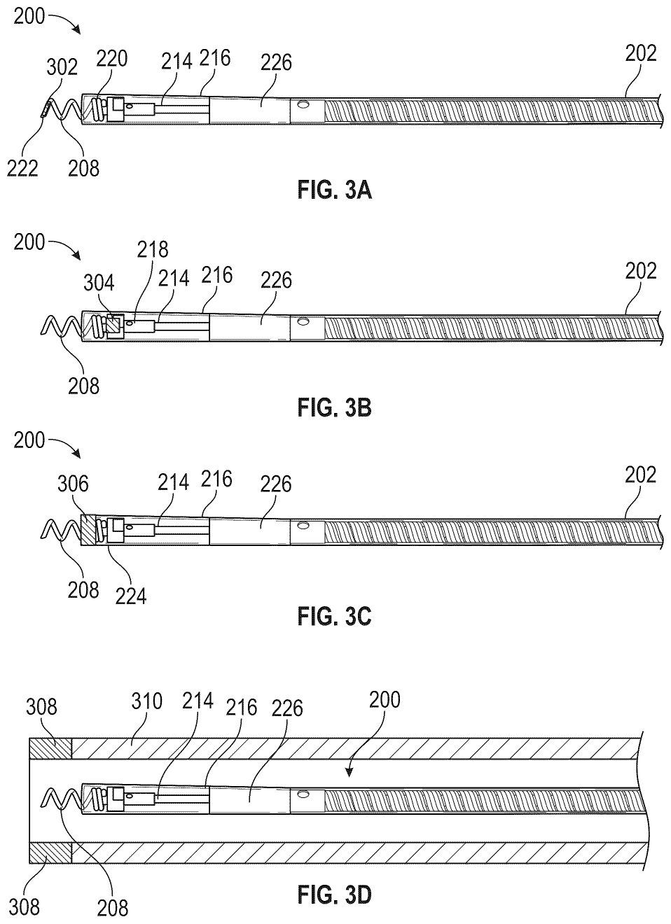

In some embodiments, the biopsy procedure involves determining the location of clinically relevant tissue in the cardiac tissue CT (e.g., pathological tissue) before obtaining a sample using the helical element 108. This can be accomplished, for example, using a sensor that senses electrical activity and/or mechanical activity of the cardiac tissue CT. The sensor may be part of the biopsy device 100 and/or another device that is operably coupled to the biopsy device 100 (e.g., a delivery catheter). Additional details of sensors that may be used are provided below, e.g., in connection with FIGS. 2-3D.

In some embodiments, the biopsy device 100 is a pacing lead. The pacing lead can be an off-the-shelf device, such as a Medtronic SelectSure MRI SureScan Model 3830 pacing lead, a Medtronic CapSureFix Novus MRI SureScan Model 5076 pacing lead, or other helix-based pacing lead. In such embodiments, the pacing lead may be used in the biopsy procedure without coupling the pacing lead to a pacemaker and/or without performing any pacing of the patient's heart. In other embodiments, however, the biopsy device 100 may be a different type of device (e.g., a device that lacks pacing functionality such as a standalone helix-based EMB tool).

FIG. 2 is a partially schematic illustration of a biopsy device 200 configured in accordance with embodiments of the present technology. The biopsy device 200 can be used to sense and/or collect a sample of tissue from an anatomic region of a patient. For example, the biopsy device 200 can be introduced intravascularly into a heart of a patient and navigated to a target region within the heart to sense and/or collect a sample of cardiac tissue therefrom (e.g., ventricular myocardium, septal wall tissue), e.g., as previously described with respect to FIG. 1.

The biopsy device 200 comprises an elongate shaft 202 with a proximal end 204 and a distal end 206, a helical element 208 at the distal end 206 of the elongate shaft 202, at least one sensor 210, and a controller 212 (shown schematically) operably coupled to the sensor 210. As described in detail below, the distal end 206 of the biopsy device 200 can be positioned proximate to cardiac tissue in a patient's heart, the sensor 210 can generate data indicative of a characteristic of the cardiac tissue, and the controller 212 can determine whether the cardiac tissue comprises pathological tissue based on the sensor data. The helical element 208 can be advanced into the cardiac tissue to collect a sample thereof, e.g., if the cardiac tissue is determined to comprise pathological tissue.

The elongate shaft 202 can be configured to be navigated through the vasculature and into the patient's heart to position the helical element 208 at a target region within the heart. In some embodiments, the elongate shaft 202 has a relatively small size (e.g., diameter) to provide improved flexibility and navigability, and/or to avoid the likelihood of inadvertent tissue trauma during introduction into the patient's body. For example, the elongate shaft 202 can have an outer diameter less than or equal to 5 mm, 4 mm, 3 mm, 2.5 mm, 2 mm, 1.5 mm, or 1 mm; and/or the outer diameter can be within a range from 1 mm to 5 mm, 1 mm to 4 mm, 1 mm to 3 mm, 1 mm to 2 mm, or 1 mm to 1.5 mm.

In some embodiments, the elongate shaft 202 is configured to be introduced into the heart via a delivery catheter that receives the elongate shaft 202. The delivery catheter may have a preset shape so that when the elongate shaft 202 is inserted into the delivery catheter, the distal end 206 of the elongate shaft 202 is positioned at a specific target region in the heart. Alternatively, the elongate shaft 202 can include a channel formed therein to accommodate a stylet or guidewire, such that the elongate shaft 202 can be advanced over the stylet/guidewire to position the distal end 206 at the target region in the heart. Optionally, the elongate shaft 202 may be steerable on its own without requiring a separate device, e.g., via steering cables within the elongate shaft 202.

In the illustrated embodiment, the elongate shaft 202 comprises an inner member 214 (e.g., an inner elongate shaft) and an outer member 216 (e.g., an outer sheath). The inner member 214 can be configured to support the helical element 208. For example, a distal end 218 of the inner member 214 can be mechanically coupled to a proximal end 220 of the helical element 208. In some embodiments, the inner member 214 can electrically couple the helical element 208 to the controller 212, e.g., in embodiments where the helical element 208 is or includes an electrical sensor such as an electrode, as discussed further below. The outer member 216 can be configured to protect the internal components of the elongate shaft 202 (e.g., the inner member 214). For example, in the illustrated embodiment, the outer member 216 has a distal end 224 that extends distally beyond the proximal end 220 of the helical element 208, thereby covering the proximal portion of the helical element 208 and the inner member 214.

Although FIG. 2 shows an embodiment of the present technology in which the elongate shaft 202 includes both the inner member 214 and the outer member 216, other configurations of the elongate shaft 202 are possible. For example, in some embodiments, the inner member 214 is omitted. In some embodiments, the outer member 216 is omitted.

The helical element 208 can be a coil, screw, spring, etc., that is located at the distal end 206 of the elongate shaft 202. The helical element 208 can be configured to be advanced partially or entirely into cardiac tissue to collect a sample thereof. For instance, the helical element 208 can be embedded into the cardiac tissue and then pulled backwards relative to the cardiac tissue (e.g., by retracting the helical element 208 and/or the elongate shaft 202 in a proximal direction) to tear out the embedded tissue. The helical element 208 may include a sharpened distal end 222 to facilitate penetration into the tissue. The geometry of the helical element 208 (e.g., length, diameter, pitch) can be varied as desired. For example, the helical element 208 can be sufficiently small to be introduced intravascularly into the heart and/or to reduce trauma to surrounding tissue. In some embodiments, the helical element 208 has an outer diameter no greater than 3 mm, 2.5 mm, 2 mm, 1.5 mm, 1.4 mm, 1.3 mm, 1.2 mm, 1.1 mm, or 1 mm; and/or within a range from 1 mm to 3 mm, 1 mm to 1.5 mm, 1.2 mm to 2 mm, or 2 mm to 3 mm. The length of the helical element 208 can be less than or equal to 5 mm, 4 mm, 3 mm, 2.5 mm, 2 mm, 1.8 mm, 1.5 mm, 1.25 mm, or 1 mm.

In some embodiments, the helical element 208 is a fixed element, e.g., the helical element 208 is not movable relative to the elongate shaft 202. In such embodiments, the helical element 208 can be translated and/or rotated with respect to the cardiac tissue by translation and/or rotation of the elongate shaft 202, respectively. The movement of the helical element 208 (e.g., translation along an axis, rotation about an axis) can facilitate sample collection. For example, the helical element 208 may be advanced distally while being rotated to embed the helical element 208 into the cardiac tissue, then pulled proximally to tear out a portion of the tissue.

In other embodiments, however, the helical element 208 can be a movable element, e.g., the helical element 208 can be configured to move with respect to the elongate shaft 202. In some embodiments, the helical element 208 can be advanced (e.g., moved distally) and/or retracted (e.g., moved proximally) with respect to the elongate shaft 202. In some embodiments, the helical element 208 is rotatable about an axis with respect to the elongate shaft 202, e.g., the helical element 208 can be spun clockwise and/or counterclockwise with respect to the elongate shaft 202. Movement of the helical element 208 relative to the elongate shaft 202 can be achieved in various ways, for example, via a twistable and/or threaded mechanism that allows the helical element 208 to be rotated relative to the elongate shaft 202, via a push/pull mechanism that allows the helical element 208 to be advanced/retracted relative to the elongate shaft 202, via an surrounding sheath or catheter that can be advanced/retracted relative to the helical element 208 to cover/expose the helical element 208, etc. Such movement may facilitate sample collection, as discussed above.

As shown in FIG. 2, the biopsy device 200 can comprise a sensor 210 configured to generate sensor data indicative of a characteristic of the cardiac tissue. For example, the sensor 210 can be configured to sense electrical activity and/or electrical properties of the heart, mechanical activity and/or mechanical properties of the heart, or both (e.g., sequentially and/or concurrently). The sensed characteristics can correlate to whether the cardiac tissue comprises pathological tissue or healthy tissue, as discussed in greater detail below.

In some embodiments, the sensor 210 of the biopsy device 200 includes an electrical sensor configured to sense electrical activity (e.g., a local or far-field electrogram (EGM), an electrocardiogram (ECG), an electromyogram (EMG)) and/or electrical properties (e.g., electrical impedance, pacing capture threshold, injury current, helix-evoked arrhythmia) of cardiac tissue. For example, the electrical sensor can include one or more electrodes that generate electrical signals that can be processed to determine electrical activity and/or electrical properties. Each electrode can be independently positioned at any suitable portion of the biopsy device 200, such as on or coupled to the distal end 222 of the helical element 208, a distal end of the elongate shaft 210 (e.g., a distal end 224 of the outer member 214), or a delivery catheter, e.g., as discussed further below with respect to FIGS. 3A-3D. Optionally, the helical element 208 itself can serve as an electrode (e.g., in embodiments where the helical element 208 is made of one or more electrically conductive materials such as metal).

In embodiments in which the electrical sensor comprises two or more electrodes, the electrodes can be positioned at the same location on the biopsy device 200 or at different locations. For example, the helical element 208 can comprise a first electrode and the elongate shaft 202 can comprise a second electrode; or the inner member 214 can comprise a first electrode and the outer member 216 can comprise a second electrode; or a first electrode can be disposed at a distal end 206 of the elongate shaft 202 and a second electrode can be disposed proximally to the distal end 206 on the elongate shaft 202; etc.

In some embodiments, the electrical sensor includes one or more electrodes (e.g., cathodes) that directly contact the cardiac tissue (e.g., ventricular myocardium, interventricular septum, etc.) to sense the electrical activity thereof (e.g., via an electrogram) or sense the electrical properties thereof (e.g., electrical impedance). Such electrode(s) can be located at the distal end 206 of the elongate shaft 202 and/or incorporated into a delivery catheter, for example. The electrical sensor can also include at least one electrode (e.g., an anode and/or return electrode) that does not directly contact cardiac tissue. Such electrode(s) can be located at portions of the elongate shaft 202 and/or delivery catheter that are spaced apart from cardiac tissue. For example, in FIG. 2, an electrode 226 is located on the elongate shaft 202, proximal to the distal end 206. In some embodiments, for example, the electrode 226 is a ring electrode. Optionally, an electrode may serve as a cathode for certain operations, and may serve as an anode and/or return electrode for other operations.

In some embodiments, the sensor 210 comprises a mechanical sensor configured to sense mechanical data (e.g., motion, mechanical properties) of the cardiac tissue of the heart. For example, the sensor 210 can be a motion sensor configured to measure beat-to-beat position, velocity, acceleration, and/or orientation of cardiac tissue to assess a characteristic of the cardiac tissue indicative of pathology (e.g., a local myocardial motion indicative of local myocardial contraction). In some embodiments, the mechanical sensor comprises one or more of a motion sensor (e.g., an accelerometer, gyroscope, inertial measurement unit (IMU)), a force sensor, a pressure sensor, or displacement sensor. In some embodiments, the helical element 208 is configured to produce a spring-like response to a push and/or pull force, by which the mechanical sensor can sense (e.g., through a strain, force, and/or pressure measurement) physical and/or mechanical information (e.g., motion, stiffness). The mechanical sensor can be positioned at any suitable portion of the biopsy device 200, such as on or coupled to the distal end 222 of the helical element 208, a distal end of the elongate shaft 210 (e.g., a distal end 224 of the outer member 214), or a delivery catheter, e.g., as discussed further below with respect to FIGS. 3A-3D.

In some embodiments, the sensor 210 can comprise another type of sensor, in addition or alternatively to electrical and mechanical sensors. For example, an optical sensor (e.g., a light fiber) can be used to obtain image data of the cardiac tissue, and the image data can be processed to determine visual characteristics indicative of pathological tissue or healthy tissue. As another example, an optical sensor or other sensor type may be used to detect local blood flow (e.g., reduced blood flow may be indicative of an ischemic region and/or other pathological change). In a further example, a chemical sensor (e.g., a myocardial glucose uptake sensor) may be used to detect chemical changes of the cardiac tissue associated with pathology.

Although FIG. 2 illustrates a single sensor 210, in other embodiments, the biopsy device 200 can include two or more sensors 210 (e.g., two sensors, three sensors, four sensors, etc.). In some embodiments, the biopsy device 200 comprises a combination of two or more sensor types, such as at least one electrical sensor and at least one mechanical sensor, at least one electrical sensor and at least one optical sensor, at least one mechanical sensor and at least one optical sensor, etc.

In the illustrated embodiment, the sensor 210 comprises the helical element 208, e.g., the entire helical element 208 serves as the sensor 210. For instance, the helical element 208 can be made of and/or be coated with an electrically conductive material (e.g., a metal such as a titanium nitride coated platinum alloy) and thus can serve as an electrode for sensing electrical activity and/or properties of cardiac tissue. In other embodiments, however, the sensor 210 can be configured differently, e.g., only a portion of the helical element 208 serves as the sensor 210, the sensor 210 is a separate component that is coupled to the helical element 208, the sensor 210 is located on the elongate shaft 202 or another device separate from the biopsy device 200, etc.

FIGS. 3A-3D are partially schematic illustrations of various sensor configurations for the biopsy device 200 of FIG. 2, in accordance with embodiments of the present technology. Any of the embodiments of FIGS. 3A-3D may be used as an alternative to or in combination with the sensor 210 illustrated in FIG. 2. Moreover, the features of the sensor 210 discussed herein with respect to FIG. 2 are also applicable to the sensors illustrated in FIGS. 3A-3D, such that the following discussion of FIGS. 3A-3D will be limited to those features that differ from the embodiment illustrated in FIG. 2.

Referring first to FIG. 3A, the biopsy device 200 can include a sensor 302 that is coupled to or part of the helical element 208. The sensor 302 can be located on only a portion of the helical element 208. For example, as shown in FIG. 3A, the sensor 302 can be coupled to or part of the distal end 222 of the helical element 208 only. This configuration can be used, for example, if the sensor 302 is configured to operate via direct contact with the cardiac tissue (e.g., via embedding into the cardiac tissue and/or contact with the surface of the cardiac tissue). In other embodiments, however, the sensor 302 can be located at any suitable portion of the helical element 208, including at the proximal end 220, or at any intermediate location between the proximal end 220 and the distal end 222.

Referring next to FIG. 3B, the biopsy device 200 can include a sensor 304 that is coupled to or part of a portion of the elongate shaft 202 supporting the helical element 208, such as the inner member 214. The sensor 304 may, for example, be coupled to or part of the distal end 218 of the inner member 214, e.g., to receive signals transmitted from the helical element 208. For example, in embodiments where the helical element 208 acts as a spring to sense mechanical properties and/or activity of the cardiac tissue, the sensor 304 can be a force transducer, strain gage, or other mechanical sensor that detects forces, strains, and/or motions of the cardiac tissue that are exerted onto the helical element 208. It will be appreciated that the sensor 304 can be coupled to or part of any portion of the inner member 214, such as a proximal end, or at any intermediate location between the distal end 218 and the proximal end.

Referring next to FIG. 3C, the biopsy device 200 can include a sensor 306 that is coupled to or part of the portion of the elongate shaft 202 surrounding the helical element 208, such as the outer member 216. As seen in FIG. 3C, in some embodiments, the sensor 306 is coupled to or part of the distal end 224 of the outer member 216. This configuration can be used, for example, in embodiments where the sensor 306 operates by being in direct contact with or in close proximity to the cardiac tissue, but does not need to be embedded into the cardiac tissue. Moreover, this configuration can be used if sensing may be performed independently of the helical element 208 being in contact with cardiac tissue, e.g., the helical element 208 can be retracted within the outer member 216 while the sensor 306 is used to obtain sensor data, and may be advanced out of the outer member 216 to embed into the tissue for sampling after it is confirmed that pathological tissue is present. It will be appreciated that the sensor 306 can be coupled to or part of any portion of the outer member 216, such as a proximal end, or at any intermediate location between the distal end 224 and the proximal end.

Although FIGS. 3B and 3C illustrate sensors 304, 306 located on the inner member 214 and the outer member 216 of the elongate shaft 202, respectively, sensors may alternatively or additionally be located on other portions of the elongate shaft 202. Moreover, other configurations of the elongate shaft 202 (e.g., without the inner member 214 or the outer member 216) can be used to support one or more sensors.

Referring next to FIG. 3D, a sensor 308 can be coupled to or part of a delivery catheter 310 having a lumen that receives the biopsy device 200 therewithin. The delivery catheter 310 can be, for example, a pacing lead delivery catheter (e.g., Medtronic C315-HIS, Medtronic C304-HIS) or another device configured to introduce the biopsy device 200 to the target region in the heart. As shown in FIG. 3D, the sensor 308 can be coupled to or part of a distal end of the delivery catheter 310. This configuration can be used, for example, in embodiments where the sensor 308 operates by being in direct contact with or in close proximity to the cardiac tissue, but does not need to be embedded into the cardiac tissue. Moreover, this configuration can be used if sensing may be performed independently of the helical element 208 being in contact with cardiac tissue, e.g., the biopsy device 200 can be retracted within the delivery catheter 310 while the sensor 308 is used to obtain sensor data, and may be advanced out of the delivery catheter 310 to embed the helical element 208 into the tissue for sampling after it is confirmed that pathological tissue is present. It will be appreciated that the sensor 308 can be coupled to or part of any portion of the delivery catheter 310, such as a proximal end, or at any intermediate location between the distal end and the proximal end.

Referring again to FIG. 2, sensing may be performed before and/or after the helical element 208 is advanced into the cardiac tissue. For instance, in embodiments where the biopsy device 200 includes a sensor that can operate without the helical element 208 being in direct contact with the cardiac tissue (e.g., the sensor 306 of FIG. 3C, the sensor 308 of FIG. 3D), the sensor may be placed in contact with the cardiac tissue to obtain sensor data thereof before the helical element 208 is advanced into the tissue. For example, if the sensor is performed using a sensor located on another component surrounding the helical element 208 and/or the biopsy device 200 (e.g., the outer member 216 or the delivery catheter 310 of FIG. 3D), sensing may be performed while the helical element 208 and/or biopsy device 200 are retracted within the component and are covered by the component. This approach may be beneficial, for instance, for avoiding injury to the tissue before it has been confirmed that the tissue is pathological and should be sampled. In other embodiments, however, even if the sensing may be performed without the helical element 208 being in direct contact with the cardiac tissue, the helical element 208 can still be advanced into contact with or embedded into the tissue before sensing is performed, e.g., to affix the position of the biopsy device 200 relative to the tissue. Optionally, the helical element 208 may initially be advanced into the tissue by a relatively small distance sufficient for fixation (e.g., to allow for easy retraction with little or no tissue damage if no pathological tissue is detected), and may be advanced further into the tissue for sampling only upon confirmation that the tissue is pathological.

As another example, in embodiments where the sensing is performed using the helical element 208 (e.g., the sensor 210 of FIG. 2, the sensor 302 of FIG. 3), the helical element 208 may be placed into contact with the tissue and/or embedded into the tissue before sensing is performed. In such embodiments, the helical element 208 may initially be advanced into the tissue by a relatively small distance sufficient for sensing (e.g., to allow for easy retraction with little or no tissue damage if no pathological tissue is detected), and may be advanced further into the tissue for sampling only upon confirmation that the tissue is pathological.

Moreover, in some embodiments, sensing may be performed using a combination of sensor types, some of which may perform sensing before the helical element 208 is advanced into the tissue and some of which may perform sensing after the helical element 208 is advanced into the tissue. For instance, a first (e.g., less accurate) sensor can obtain sensor data before the helical element 208 is placed into contact with and/or embedded into the tissue. If the sensor data indicates that the tissue is likely to be pathological, the helical element 208 can then be advanced into the tissue and a second (e.g., more accurate) sensor can obtain sensor data to confirm that the tissue is indeed pathological and should be sampled. This approach may be used to reduce the likelihood of damaging healthy tissue while also providing high accuracy detection of pathological tissue.

The controller 212 (which may also be referred to herein as an “analyzer”) is operably coupled to the sensor 210 to determine whether the cardiac tissue comprises pathological tissue or healthy tissue, based on the sensor data. For example, the controller 212 can be electrically coupled to the sensor 210 via a connection 228 at the proximal end 204 of the elongate shaft 202. The connection 228 may be a wired connection as shown in FIG. 2 (e.g., one or more cables), a wireless connection (e.g., BLUETOOTH®, Wi-Fi, Medical Implant Communication Service (MICS)), or a combination thereof. The controller 212 can be or include a computing device including one or more processors, memory, output devices (e.g., a display), input devices (e.g., keyboard, mouse, touchscreen), and/or other hardware and/or software components suitable for effectuating the operations described herein. The controller 212 may be a standalone device or may be remotely controlled (e.g., by another computing device in communication with the controller 212).

The processor of the controller 212 can include a microprocessor, a controller, a digital signal processor (DSP), an application specific integrated circuit (ASIC), a field-programmable gate array (FPGA), or equivalent discrete or analog logic circuitry. In some embodiments, the processor can include multiple components, such as any combination of one or more microprocessors, controllers, DSPs, ASICs, and/or FPGAs, as well as other discrete or integrated logic circuitry. The functions attributed to the processor herein may be embodied as software, firmware, hardware, or any combination thereof.

The memory (e.g., a data storage device or other non-transitory medium) of the controller 212 can store computer-readable instructions that, when executed by the processor, cause the biopsy device 200 to perform various operations described herein. The memory can include any volatile, non-volatile, magnetic, optical, or electrical media, such as a random-access memory (RAM), read only memory (ROM), non-volatile RAM (NVRAM), electrically-erasable programmable ROM (EEPROM), flash memory, or any other digital or analog media.

The display of the controller 212 can display information relating to and/or received from the biopsy device 200, such as intracardiac electrogram (EGM) and/or other electrical signals obtained by the biopsy device 200, motion sensor signals and/or other mechanical data acquired by the biopsy device 200, operational parameters of the biopsy device 200, etc. In some embodiments, the controller 212 is configured to display instructions to a user of the biopsy device 200, e.g., instructions to obtain a biopsy sample, instructions to reposition the biopsy device 200 to another location, etc.

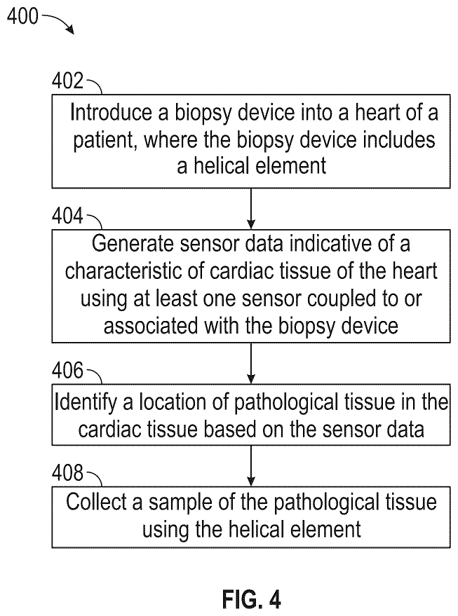

The controller 212 can implement a software algorithm that analyzes the sensor data (e.g., electrical signals, mechanical data) generated by sensor 210. Based on the analysis of sensor data, the algorithm can determine whether cardiac tissue is suitable for biopsy (e.g., whether the cardiac tissue comprises pathological tissue) and can output instructions to the user (e.g., to obtain a biopsy sample from the current location if a pathological tissue is detected at that location or to reposition the biopsy device 200 to a different location if pathological tissue is not detected at the current location). Further details of procedures and algorithms that may be performed by the controller 212 of the biopsy device 200 are shown and described below in connection with FIGS. 4-7.

FIG. 4 is a flow diagram illustrating a method 400 for collecting a biopsy sample from a patient, in accordance with embodiments of the present technology. The method 400 can be performed using any of the devices described herein, e.g., with respect to FIGS. 1-3D above. In some embodiments, at least some of the processes of the method 400 are implemented as computer-readable instructions (e.g., program code) that are configured to be executed by one or more processors (e.g., one or more processors of the controller 212 of the biopsy device 200 of FIG. 2).

At block 402, the method 400 can begin with introducing a biopsy device into a heart of a patient (e.g., the biopsy device 100 of FIG. 1 or the biopsy device 200 of FIGS. 2-3D). For example, the biopsy device can be inserted into the vasculature through an insertion point and navigated into a chamber of the heart (e.g., the right atrium, the right ventricle, the left ventricle, or the left atrium), such that a distal end of the biopsy device is placed proximate to a target region in the heart containing cardiac tissue of interest (e.g., ventricular myocardium, septal wall tissue). The biopsy device may be navigated to the target region on its own or with aid from another device (e.g., a delivery catheter, sheath, guidewire, stylet).

In some embodiments, the biopsy device includes a helical element (e.g., the helical element 108 of FIG. 1 or the helical element 208 of FIGS. 2-3D). The helical element can be located at the distal end of the biopsy device and thus can be introduced to the target region by the biopsy device. In some embodiments, the process of block 402 includes advancing the helical element toward the cardiac tissue such that the helical element touches the surface of the tissue and/or penetrates into the tissue, e.g., to affix the biopsy device to the tissue and/or to facilitate sensing, as discussed herein. In other embodiments, the helical element is not brought into contact with the tissue during the process of block 402, e.g., the helical element may remain retracted within the biopsy device and/or within another device that receives the biopsy device.

At block 404, the method 400 can include generating sensor data indicative of a characteristic of cardiac tissue of the heart using at least one sensor coupled to or associated with the biopsy device (e.g., the sensor 210 of FIG. 2, the sensor 302 of FIG. 3A, the sensor 304 of FIG. 3B, the sensor 306 of FIG. 3C, and/or the sensor 308 of FIG. 3D). The sensor can be configured to sense electrical characteristics, mechanical characteristics, visual characteristics, and/or other characteristics of interest that may be relevant to assessing the patient's condition. For example, the sensor can be or include an electrical sensor (e.g., one or more electrodes) configured to sense electrical activity (e.g., an EGM, ECG, EMG) and/or electrical properties (e.g., electrical impedance, pacing capture threshold, injury current, helix-evoked arrhythmia) of cardiac tissue. As another example, the sensor can be or include a mechanical sensor (e.g., a motion sensor, force sensor, pressure sensor, displacement sensor) configured to sense motion and/or mechanical properties of the heart. Other types of sensors may alternatively or additionally be used, such as optical sensors, blood flow sensors, chemical sensors, etc. The sensor can be located at any suitable location on the biopsy device (e.g., the helical element, an elongate shaft supporting the helical element) and/or on another device associated with the biopsy device (e.g., a delivery catheter for the biopsy device).

As described elsewhere herein, sensing may be performed with or without the helical element being advanced into the cardiac tissue. In some embodiments, sensing is performed while the helical element is in contact with the cardiac tissue but does not penetrate into the cardiac tissue. In some embodiments, sensing is performed while the helical element is embedded into the cardiac tissue. In some embodiments, sensing is performed while the helical element is retracted away from the cardiac tissue.

At block 406, the method 400 can continue with identifying a location of pathological tissue in the cardiac tissue based on the sensor data. The process of block 406 may be performed using a software algorithm (e.g., a rule-based algorithm, a machine learning algorithm) that is implemented by a computing device (e.g., the controller 212 of FIG. 2). The algorithm can be configured to analyze the sensor data obtained at a location in the heart to determine whether the sensor data indicates the presence of pathological tissue at that location. Specifically, electrical signals, mechanical data, and/or other information from the sensor data can be analyzed to identify values, waveforms, patterns, and/or other signal characteristics that are correlated with pathological tissue versus healthy tissue. Pathological tissue that may be detected using the techniques herein include but are not limited to tissue with significant fibrosis, tissue that exhibits abnormal (e.g., weakened) contractility, tissue of the heart with abnormal material composition (e.g., cardiac amyloidosis, iron-overload cardiomyopathy, glycogen storage disease), cardiac tumor, inflamed tissue (e.g., tissue with myocarditis, tissue with infection), tissue from a target region that exhibits abnormal electrical activity (e.g., arrhythmia, bradycardia, tachycardia, premature beats, sick sinus syndrome), ischemic tissue, tissue from a target region that exhibits abnormal biochemical activity, and/or tissue from a target region that exhibits abnormal mechanical activity, such as an atypical venous pressure waveform (due to, e.g., atrial flutter, tricuspid valve stenosis, tricuspid insufficiency, pericardial constriction, tamponade).

In some embodiments, one or more values associated with the sensor data may be used to distinguish healthy tissue from pathological tissue. In some embodiments, one or more values from the sensor data may be compared to a threshold value (e.g., a value stored in the memory of the controller 212), where the sensed value(s) being above or below the threshold value indicates possible pathology of cardiac tissue sensed by the sensor. For example, in embodiments in which a mechanical sensor is used to measure tissue stiffness, tissue stiffness above or below an acceptable threshold value may be indicative of pathological tissue. As another example, in embodiments in which a mechanical sensor is used to measure mechanical motion or force, amplitude or force values below a threshold value may indicate weakening of cardiac tissue indicative of fibrosis and/or other pathologies.

In some embodiments, the sensor data includes or is used to generate a waveform representing cardiac activity (e.g., local electrical and/or mechanical activity), and the waveform is analyzed to detect characteristics indicative of pathological cardiac activity. The analysis may be performed, for example, by comparing the waveform to a template waveform (e.g., a predetermined waveform that is known to represent pathological or non-pathological characteristics), by comparing the waveform to a threshold (e.g., to determine whether the waveform is above or below the threshold, to determine a duration that the waveform is within an acceptable range versus an unacceptable range), by measuring one or more waveform characteristics (e.g., width, morphology, amplitude, frequency, duty cycle), etc. For instance, a template electrical waveform characteristic of normal, non-pathological cardiac muscle activity (e.g., a QRS complex) can be obtained, and deviations from that template waveform as reported by the sensor data can be used to distinguish non-pathological tissue from pathological tissue. As another example, a template waveform characteristic of pathological cardiac muscle activity (e.g., (e.g., a fragmented QRS complex) can be obtained, and similarities to that template waveform as reported by the sensor data can be used to identify pathological tissue. Electrical waveform characteristics that may be indicative of pathology include notches in EGM, wide local EGM, low amplitude EGM, small d/dtₘₐₓ of the EGM upstroke, high pacing capture threshold (e.g., greater than 2 volts at 0.5 milliseconds), a premature ventricular contraction, etc.

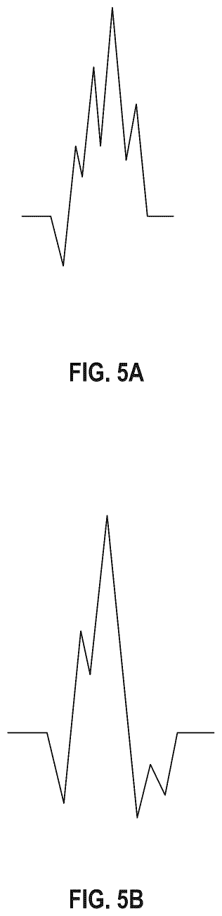

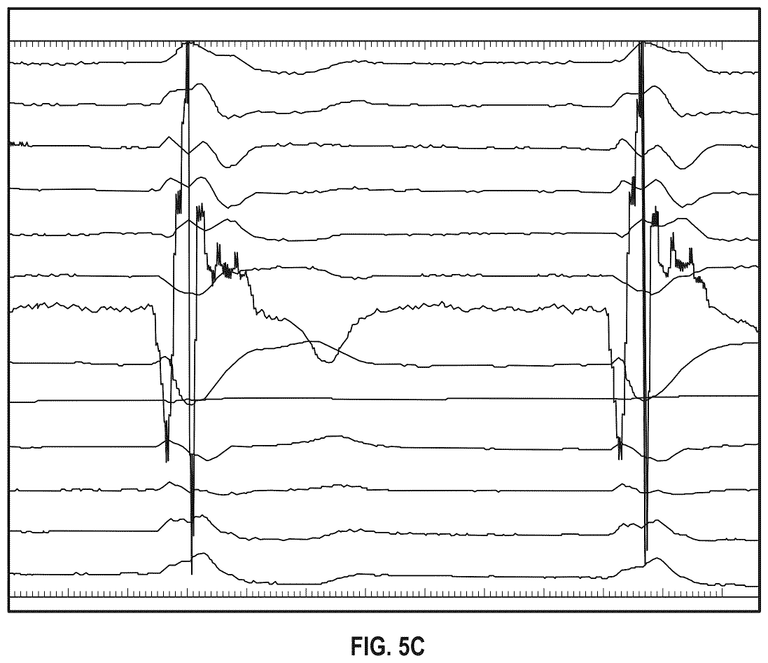

FIGS. 5A-5C illustrate representative examples of waveforms corresponding to pathological cardiac activity, in accordance with embodiments of the present technology. For example, FIGS. 5A and 5B illustrate examples of fragmented QRS morphology (fQRS) which may be indicative of scarring and/or other pathological changes. FIG. 5C illustrates a fQRS recorded at the interventricular septum in a patient with interventricular conduction delay. Presence of an fQRS waveform can signify myocardial scarring in patients (e.g., in patients with coronary artery disease, in patients with nonischemic dilated cardiomyopathy) due to resultant myocardial conduction abnormalities. Furthermore, the presence of the fQRS waveform can contribute to predicting arrhythmic event(s) in a patient.

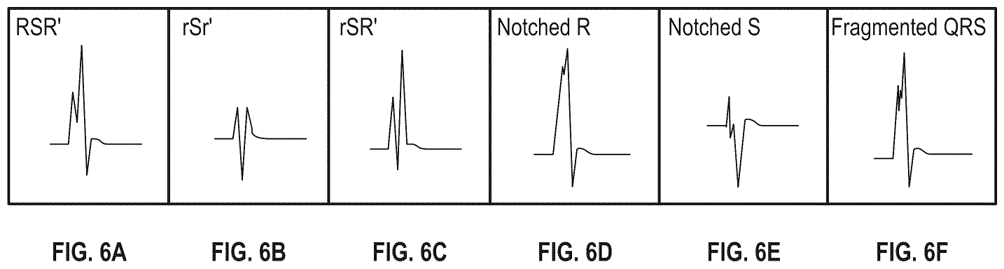

FIGS. 6A-6F illustrate representative examples of fQRS waveforms corresponding to pathological cardiac activity, in accordance with embodiments of the present technology. FIG. 6A illustrates an example fQRS waveform referred to as an RSR′ pattern. The RSR′ pattern includes various morphologies of the QRS interval with or without the Q wave and comprises an additional R wave (R′), notching in the nadir of the S wave, and/or the presence of more than one R′ (fragmentation) in two contiguous waveforms. FIG. 6B illustrates an example fQRS waveform referred to as an rSr′ pattern. The rSr′ pattern includes an initially small positive R wave (r), followed by a large negative wave (an S wave), and an additional small R wave (r′). FIG. 6C illustrates an example fQRS waveform referred to as an rSR′ pattern. The rSR′ pattern includes an initially small positive R wave (r), followed by a negative wave (an S wave), and an additional R wave (R′). In some instances, the wave following the initially small positive R wave is more negative than the peak value of the R wave (e.g., is lesser in value), but does not pass a baseline value. FIG. 6D illustrates an example fQRS waveform referred to as a notched R pattern. The notched R pattern includes a deviation from a typical R wave, such as a notching (e.g., a significant short-term deviation from a normal value) on an upstroke of the R wave or a notching on a downstroke of the R wave. FIG. 6E illustrates an example fQRS waveform referred to as a notched S waveform. The notched S waveform includes a deviation from a typical S wave, such as a notching on the downstroke of the S wave, a notching at the nadir of the S wave, and/or a notching on the upstroke of the S wave. FIG. 6F illustrates an example fQRS waveform comprising multiple fQRS morphologies. By analyzing sensor data for an fQRS waveform, cardiac tissue can be determined to comprise pathological tissue or healthy tissue.

Referring again to FIG. 4, various types of software algorithms can be used to analyze the sensor data to determine whether the sensed cardiac tissue is pathological tissue or healthy tissue. Such algorithms may include rule-based algorithms, machine learning algorithms, or suitable combinations thereof. For instance, a machine learning algorithm can be trained on previous patient data, experimental data, literature data, etc., to classify a waveform as pathological or non-pathological. The inputs to the software algorithm may optionally include other clinically relevant data besides the sensor data, such as demographic information (e.g., age, gender, ethnicity), medical history (e.g., known cardiac conditions, comorbidities, diagnoses), other data characterizing the cardiac tissue (e.g., echocardiography data), etc.

Optionally, the process of block 406 can further include outputting a notification to a user, based on the analysis of the sensor data. For instance, if the analysis indicates that pathological tissue is present at the particular location, the notification can instruct the user to collect a sample of the tissue using the biopsy device. Conversely, if the analysis indicates that pathological tissue is not present at the particular location, the notification can instruct the user not to collect a sample of the tissue and/or to reposition the biopsy device to a different location. Other types of information can also be provided, such as the raw and/or processed sensor data (e.g., so the user can form their own assessment of whether pathological tissue is present), type of pathology detected (if applicable), recommended actions (e.g., suggestions on how to reposition the biopsy device), information regarding the operational status of the biopsy device, etc. Notifications may be presented to the user on a display (e.g., the display of the controller 212 of FIG. 2) and may be presented in any suitable format (e.g., text, graphics, audio alerts, haptic feedback, lights or other visual indicators).

At block 408, the method 400 can include collecting a sample of the pathological tissue using the helical element. The collection may be performed, for example, if the process of block 406 indicates that pathological tissue is present at the sensed location. In some embodiments, the collection is performed by advancing the helical element of the biopsy device into the cardiac tissue so as to penetrate into the tissue, and then retracting the helical element and/or biopsy device relative to the cardiac tissue to pull out a sample of the tissue. In some embodiments, the helical element of the biopsy device is already advanced into the cardiac tissue prior to the process of block 408, and the collection process involves retracting the helical element and/or biopsy device relative to the cardiac tissue to pull out a sample of the tissue. In some embodiments, the helical element of the biopsy device is advanced partially into the cardiac tissue prior to the process of block 408, and the collection process involves advancing the helical element further into the cardiac tissue and then retracting the helical element and/or biopsy device relative to the cardiac tissue to pull out a sample of the tissue.

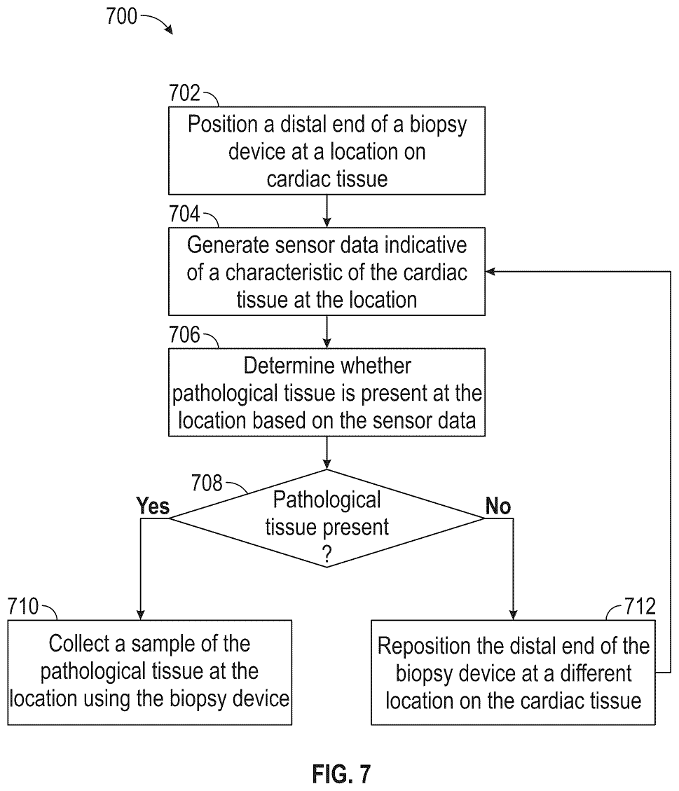

FIG. 7 is a flow diagram illustrating a method 700 for collecting a biopsy sample from a patient, in accordance with embodiments of the present technology. The method 700 can be performed using any of the devices described herein, e.g., with respect to FIGS. 1-3D above. The method 700 can be combined with any of the other methods described herein, such as the method 400 of FIG. 4. For example, the method 700 can be performed as part of the process of block 406 of the method 400 of FIG. 4 to identify a location of pathological tissue based on sensor data. In some embodiments, at least some of the processes of the method 700 are implemented as computer-readable instructions (e.g., program code) that are configured to be executed by one or more processors (e.g., one or more processors of the controller 212 of the biopsy device 200 of FIG. 2).

The method 700 can begin at block 702 with positioning a distal end of a biopsy device at a location on cardiac tissue. The process of block 702 may be identical or generally similar to the process of block 402 of the method 400 of FIG. 4. For example, the biopsy device can be any of the embodiments described herein, such as the biopsy device 100 of FIG. 1 or the biopsy device 200 of FIGS. 2-3D. The biopsy device can be inserted into the vasculature through an insertion point and navigated into a chamber of the heart, such that the distal end of the biopsy device is placed proximate to the location on the cardiac tissue. The biopsy device may be navigated to the location on its own or with aid from another device.

In some embodiments, the biopsy device includes a helical element (e.g., the helical element 108 of FIG. 1 or the helical element 208 of FIGS. 2-3D). The helical element can be located at the distal end of the biopsy device and thus can be introduced to the location on the cardiac tissue by the biopsy device. In some embodiments, the process of block 702 includes advancing the helical element toward the location on the cardiac tissue such that the helical element touches the surface of the tissue and/or penetrates into the tissue. In other embodiments, the helical element is not brought into contact with the cardiac tissue during the process of block 702.

At block 704, the method 700 can include generating sensor data indicative of a characteristic of the cardiac tissue at the location. The process of block 704 may be identical or generally similar to the process of block 404 of the method 400 of FIG. 4. For example, the sensor data can be generated using any of the sensors described herein, such as the sensor 210 of FIG. 2, the sensor 302 of FIG. 3A, the sensor 304 of FIG. 3B, the sensor 306 of FIG. 3C, and/or the sensor 308 of FIG. 3D. The sensor can be configured to sense electrical characteristics, mechanical characteristics, visual characteristics, and/or other characteristics of interest that may be relevant to assessing the patient's condition. The sensor can be located at any suitable location on the biopsy device and/or on another device associated with the biopsy device. As described elsewhere herein, sensing may be performed with or without the helical element being advanced into the cardiac tissue. In some embodiments, sensing is performed while the helical element is in contact with the cardiac tissue but does not penetrate into the cardiac tissue. In some embodiments, sensing is performed while the helical element is embedded into the cardiac tissue. In some embodiments, sensing is performed while the helical element is retracted away from the cardiac tissue.

At block 706, the method 700 can continue with determining whether pathological tissue is present at the location based on the sensor data. The process of block 706 may be performed using a software algorithm (e.g., a rule-based algorithm, a machine learning algorithm) implemented by a computing device (e.g., the controller 212 of FIG. 2), e.g., as previously discussed with respect to block 406 of the method 400 of FIG. 4. For example, the algorithm can be configured to analyze the sensor data obtained at a location in the heart to determine the sensor data indicates the presence of pathological tissue at that location. Specifically, electrical signals, mechanical data, and/or other information from the sensor data can be analyzed to identify values, waveforms, patterns, and/or other signal characteristics that are correlated with pathological tissue versus healthy tissue. In some embodiments, the sensor data includes or is used to generate a waveform representing cardiac activity, and the waveform is analyzed to detect characteristics indicative of pathological cardiac activity. The analysis may be performed, for example, by comparing the waveform to a template waveform, by comparing the waveform to a threshold, by measuring one or more waveform characteristics, or suitable combinations thereof.

At block 708, the method 700 can evaluate whether pathological tissue was determined to be present. If pathological tissue was determined to be present at the location, the method 700 can proceed to block 710 with collecting a sample of the pathological tissue at the location using the biopsy device. The process of block 710 may be identical or generally similar to the process of block 408 of the method 400 of FIG. 4. For example, the collection can be performed using the helical element of the biopsy device, such as by advancing the helical element into the cardiac tissue so as to penetrate into the tissue, and then retracting the helical element and/or biopsy device relative to the cardiac tissue to pull out a sample of the tissue. In some embodiments, the helical element of the biopsy device is already advanced into the cardiac tissue prior to the process of block 708, and the collection process involves retracting the helical element and/or biopsy device relative to the cardiac tissue to pull out a sample of the tissue. In some embodiments, the helical element of the biopsy device is advanced partially into the cardiac tissue prior to the process of block 708, and the collection process involves advancing the helical element further into the cardiac tissue and then retracting the helical element and/or biopsy device relative to the cardiac tissue to pull out a sample of the tissue.

If pathological tissue was determined to not be present at the location, the method 700 can instead proceed to block 712 with repositioning the distal end of the biopsy device at a different location on the cardiac tissue. The biopsy device may be repositioned to the different location on its own or with aid from another device (e.g., a delivery catheter, sheath, stylet, guidewire). The method 700 can then repeat the processes of blocks 704-708 to determine whether pathological tissue is present at the new location. The repositioning, sensing, and determining can be iterated until a location containing pathological tissue is identified.

Optionally, the method 700 can further include outputting notifications to a user to provide guidance in collecting the sample and/or repositioning the biopsy device. For instance, if the process of block 706 determines that pathological tissue is present at the location, the notification can instruct the user to collect a sample of the tissue using the biopsy device prior to the process of block 710. Conversely, if the process of block 706 determines that pathological tissue is not present at the location, the notification can instruct the user to reposition the biopsy device to a different location prior to the process of block 712. Other types of information can also be provided, such as the raw and/or processed sensor data (e.g., so the user can form their own assessment of whether pathological tissue is present), type of pathology detected (if applicable), recommended actions (e.g., suggestions on how to reposition the biopsy device), information regarding the operational status of the biopsy device, etc. Notifications may be presented to the user on a display (e.g., the display of the controller 212 of FIG. 2) and may be presented in any suitable format (e.g., text, graphics, audio alerts, haptic feedback, lights or other visual indicators).

Although certain embodiments of the present technology are described with respect to differentiating pathological tissue from healthy tissue, this is not intended to be limiting, and the techniques herein may be applied to other approaches for identifying tissue to be biopsied. For instance, the devices and methods herein can alternatively or additionally be used to distinguish different tissue types (any of which may or may not be pathological), where a first tissue type is of interest for biopsy purposes and a second tissue type is not of interest for biopsy purposes. Moreover, in some instances, sampling of healthy tissue may be performed, e.g., to provide baseline information on the patient's condition and/or to provide a reference for comparison to pathological tissue.

### EXAMPLES

The following examples are included to further describe some aspects of the present technology, and should not be used to limit the scope of the technology.

Example 1. A device for collecting a biopsy sample, the device comprising:
an elongate shaft comprising a proximal end and a distal end, wherein the distal end is configured to be introduced into a heart of a patient and positioned proximate to cardiac tissue of the heart;
a helical element at the distal end of the elongate shaft, wherein the helical element is configured to be advanced into the cardiac tissue;
at least one sensor configured to generate sensor data indicative of a characteristic of the cardiac tissue; and
a controller comprising one or more processors and a memory storing instructions that, when executed by the one or more processors, cause the controller to perform operations comprising:
   receiving the sensor data from the at least one sensor,
   determining whether the cardiac tissue comprises pathological tissue based on the sensor data,
   if the cardiac tissue comprises pathological tissue, instructing a user to collect a sample of the pathological tissue using the helical element, and
   if the cardiac tissue does not comprise pathological tissue, instructing the user to reposition the distal end of the elongate shaft.

Example 2. The device of Example 1, wherein the helical element has an outer diameter no greater than 3 mm.

Example 3. The device of Example 1 or 2, wherein the helical element is fixed.

Example 4. The device of Example 1 or 2, wherein the helical element is retractable.

Example 5. The device of any one of Examples 1 to 4, wherein the at least one sensor comprises the helical element or a sensor located on the helical element.

Example 6. The device of any one of Examples 1 to 5, wherein the at least one sensor comprises a sensor located on the elongate shaft.

Example 7. The device of any one of Examples 1 to 6, further comprising a delivery catheter comprising a lumen configured to receive the elongate shaft, wherein the at least one sensor comprises a sensor located on the delivery catheter.

Example 8. The device of any one of Examples 1 to 7, wherein the at least one sensor comprises an electrical sensor and the sensor data comprises electrical signals of the cardiac tissue.

Example 9. The device of Example 8, wherein the electrical sensor comprises one or more electrodes.

Example 10. The device of any one of Examples 1 to 9, wherein the at least one sensor comprises a mechanical sensor and the sensor data comprises mechanical data of the cardiac tissue.

Example 11. The device of Example 10, wherein the mechanical sensor comprises one or more of a motion sensor, a force sensor, a pressure sensor, or a displacement sensor.

Example 12. The device of any one of Examples 1 to 11, wherein the sensor data comprises a waveform indicative of the characteristic of the cardiac tissue, and the determination is based on a comparison of the waveform to a template waveform, a comparison of the waveform to a threshold, a width of the waveform, a morphology of the waveform, or a combination thereof.

Example 13. The device of any one of Examples 1 to 12, wherein the at least one sensor is configured to generate the sensor data after the helical element has been advanced into the cardiac tissue.

Example 14. The device of any one of Examples 1 to 12, wherein the at least one sensor is configured to generate the sensor data before the helical element has been advanced into the cardiac tissue.

Example 15. A method for collecting a biopsy sample, the method comprising:
introducing a biopsy device into a heart of a patient, wherein the biopsy device comprises an elongate shaft having a distal end and a helical element at the distal end;
generating sensor data indicative of a characteristic of cardiac tissue of the heart using at least one sensor coupled to or associated with the biopsy device;
identifying a location of pathological tissue in the cardiac tissue based on the sensor data; and
collecting a sample of the pathological tissue using the helical element.

Example 16. The method of Example 15, further comprising:
positioning the distal end of the elongate shaft at a first location on the cardiac tissue,
generating sensor data indicative of the characteristic of the cardiac tissue at the first location,
determining whether the pathological tissue is present at the first location based on the sensor data,
if the pathological tissue is present at the first location, collecting a sample of the pathological tissue at the first location using the helical element, and
if the pathological tissue is not present at the first location, repositioning the distal end of the elongate shaft at a second location on the cardiac tissue.

Example 17. The method of Example 15 or 16, wherein the helical element has an outer diameter no greater than 3 mm.

Example 18. The method of any one of Examples 15 to 17, wherein the helical element is fixed.

Example 19. The method of any one of Examples 15 to 17, wherein the helical element is retractable.

Example 20. The method of any one of Examples 15 to 19, wherein the at least one sensor comprises the helical element or a sensor located on the helical element.

Example 21. The method of any one of Examples 15 to 20, wherein the at least one sensor comprises a sensor located on the elongate shaft.

Example 22. The method of any one of Examples 15 to 21, wherein the biopsy device is introduced into the heart via a delivery catheter, and wherein the at least one sensor comprises a sensor located on the delivery catheter.

Example 23. The method of any one of Examples 15 to 22, wherein the at least one sensor comprises an electrical sensor and the sensor data comprises electrical signals of the cardiac tissue.

Example 24. The method of Example 23, wherein the electrical sensor comprises one or more electrodes.

Example 25. The method of any one of Examples 15 to 24, wherein the at least one sensor comprises a mechanical sensor and the sensor data comprises mechanical data of the cardiac tissue.

Example 26. The method of Example 25, wherein the mechanical sensor comprises one or more of a motion sensor, a force sensor, a pressure sensor, or a displacement sensor.

Example 27. The method of any one of Examples 15 to 26, wherein the sensor data comprises a waveform indicative of the characteristic of the cardiac tissue, and the method further comprises comparing the waveform to a template waveform, comparing the waveform to a threshold, determining a width of the waveform, determining a morphology of the waveform, or a combination thereof.

Example 28. The method of any one of Examples 15 to 27, further comprising advancing the helical element into the cardiac tissue before generating the sensor data.

Example 29. The method of any one of Examples 15 to 27, further comprising advancing the helical element into the cardiac tissue after generating the sensor data.

### CONCLUSION

Although many of the embodiments are described above with respect to systems, devices, and methods for cardiac tissue biopsy, the technology is applicable to other applications and/or other approaches, such as other biopsy of tissues of other anatomical regions (e.g., the liver). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-7.

The embodiments of the present technology can be implemented, at least in part, in hardware, software, firmware or any combination thereof. For example, various embodiments can be implemented within one or more processors, including one or more microprocessors, DSPs, ASICS, FPGAs, or any other equivalent integrated or discrete logic circuitry, as well as any combinations of such components, embodied in programmers (e.g., physician or patient programmers), stimulators, or other devices. The terms “processor” or “processing circuitry” may generally refer to any of the foregoing logic circuitry, alone or in combination with other logic circuitry, or any other equivalent circuitry.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A device for collecting a biopsy sample, the device comprising:
an elongate shaft comprising a proximal end and a distal end, wherein the distal end is configured to be introduced into a heart of a patient and positioned proximate to cardiac tissue of the heart;
a helical element at the distal end of the elongate shaft, wherein the helical element is configured to be advanced into the cardiac tissue;
at least one sensor configured to generate sensor data indicative of a characteristic of the cardiac tissue; and
a controller comprising one or more processors and a memory storing instructions that, when executed by the one or more processors, cause the controller to perform operations comprising:
receiving the sensor data from the at least one sensor,
determining whether the cardiac tissue comprises pathological tissue based on the sensor data,
if the cardiac tissue comprises pathological tissue, instructing a user to collect a sample of the pathological tissue using the helical element, and
if the cardiac tissue does not comprise pathological tissue, instructing the user to reposition the distal end of the elongate shaft.

2. The device of claim 1, wherein the helical element has an outer diameter no greater than 3 mm.

3. The device of claim 1, wherein the helical element is fixed.

4. The device of claim 1, wherein the helical element is retractable.

5. The device of claim 1, wherein the at least one sensor comprises the helical element or a sensor located on the helical element.

6. The device of claim 1, wherein the at least one sensor comprises a sensor located on the elongate shaft.

7. The device of claim 1, further comprising a delivery catheter comprising a lumen configured to receive the elongate shaft, wherein the at least one sensor comprises a sensor located on the delivery catheter.

8. The device of claim 1, wherein the at least one sensor comprises an electrical sensor and the sensor data comprises electrical signals of the cardiac tissue.

9. The device of claim 8, wherein the electrical sensor comprises one or more electrodes.

10. The device of claim 1, wherein the at least one sensor comprises a mechanical sensor and the sensor data comprises mechanical data of the cardiac tissue.

11. The device of claim 10, wherein the mechanical sensor comprises one or more of a motion sensor, a force sensor, a pressure sensor, or a displacement sensor.

12. The device of claim 1, wherein the sensor data comprises a waveform indicative of the characteristic of the cardiac tissue, and the determination is based on a comparison of the waveform to a template waveform, a comparison of the waveform to a threshold, a width of the waveform, a morphology of the waveform, or a combination thereof.

13. The device of claim 1, wherein the at least one sensor is configured to generate the sensor data after the helical element has been advanced into the cardiac tissue.

14. The device of claim 1, wherein the at least one sensor is configured to generate the sensor data before the helical element has been advanced into the cardiac tissue.

15. A method for collecting a biopsy sample, the method comprising:
introducing a biopsy device into a heart of a patient, wherein the biopsy device comprises an elongate shaft having a distal end and a helical element at the distal end;
generating sensor data indicative of a characteristic of cardiac tissue of the heart using at least one sensor coupled to or associated with the biopsy device;
identifying a location of pathological tissue in the cardiac tissue based on the sensor data; and
collecting a sample of the pathological tissue using the helical element.

16. The method of claim 15, further comprising:
positioning the distal end of the elongate shaft at a first location on the cardiac tissue,
generating sensor data indicative of the characteristic of the cardiac tissue at the first location,
determining whether the pathological tissue is present at the first location based on the sensor data,
if the pathological tissue is present at the first location, collecting a sample of the pathological tissue at the first location using the helical element, and
if the pathological tissue is not present at the first location, repositioning the distal end of the elongate shaft at a second location on the cardiac tissue.

17. The method of claim 15, wherein the helical element has an outer diameter no greater than 3 mm.

18. The method of claim 15, wherein the at least one sensor comprises the helical element or a sensor located on the helical element.

19. The method of claim 15, wherein the biopsy device is introduced into the heart via a delivery catheter, and wherein the at least one sensor comprises a sensor located on the delivery catheter.

20. The method of claim 15, wherein the at least one sensor comprises an electrical sensor and the sensor data comprises electrical signals of the cardiac tissue.
